(19) United States
(12) Patent Application Publication
(10) Pub. No.: US 2002/0161326 A1
(43) Pub. Date: Oct. 31, 2002

(54) TIP FOR A LIQUEFRACTURE HANDPIECE

(76) Inventors: GLENN SUSSMAN, LAKE FOREST, CA (US); THOMAS G. CAPETAN, FORT WORTH, TX (US); DONALD M. COHEN, IRVINE, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/428,978

(22) Filed: Oct. 28, 1999

Related U.S. Application Data
(63) Continuation-in-part of application No. 09/130,131, filed on Aug. 6, 1998, now Pat. No. 5,997,499.
Application No. 09/130,131 is a continuation-in-part of application No. 09/090,433, filed on Jun. 4, 1998, now Pat. No. 6,080,128.

(51) Int. Cl. A61M 1/00

(52) U.S. Cl. 604/35; 604/27

(57) ABSTRACT

A tip for a liquefraction surgical handpiece. The tip uses at least two channels or tubes. One tube is used for aspiration and at least one other tube is used to inject heated surgical fluid for liquefying a cataractous lens. The distal portion of the injection tube terminates just inside of the aspiration tube and can be steered or directed so that heated fluid escaping the injection tube can be injected directly into the eye or reflected off of the internal wall of the aspiration tube prior to entering the eye. Alternatively, the injection tube may be relatively rigid but be extendable or contain a rotatable deflector that alternatively allows the heated fluid to be injected directly into the eye or reflected off of the internal wall of the aspiration tube. The handpiece may also contain other tubes, for example, for injecting relatively cool surgical fluid.

This application is a continuation-in-part application of U.S. patent application Ser. No. 09/130,131, filed Aug. 6, 1998, which was a continuation-in-part application of U.S. patent application Ser. No. 09/090,433, filed Jun. 4, 1998.

### BACKGROUND OF THE INVENTION

This invention relates generally to the field of cataract surgery and more particularly to a handpiece for practicing the liquefracture technique of cataract removal.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4,246,902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted with the injection of the heated solution and the introduction of a relatively cool irrigating solution, thereby quickly cooling and removing the heated solution. This technique is more fully described in U.S. Pat. No. 5,616,120 (Andrew, et al.), the entire contents of which is incorporated herein by reference. The apparatus disclosed in the publication, however, heats the solution separately from the surgical handpiece. Temperature control of the heated solution can be difficult because the fluid tubings feeding the handpiece typically are up to two meters long, and the heated solution can cool considerably as it travels down the length of the tubing.

Therefore, a need continues to exist for a surgical handpiece that can heat internally the solution used to perform the liquefracture technique.

### BRIEF SUMMARY OF THE INVENTION

The present invention improves upon the prior art by providing a tip for a liquefraction surgical handpiece. The tip uses at least two channels or tubes. One tube is used for aspiration and at least one other tube is used to inject heated surgical fluid for liquefying a cataractous lens. The distal portion of the injection tube terminates just inside of the aspiration tube and can be steered or directed so that heated fluid escaping the injection tube can be injected directly into the eye or reflected off of the internal wall of the aspiration tube prior to entering the eye. Alternatively, the injection tube may be relatively rigid but be extendable or contain a rotatable deflector that alternatively allows the heated fluid to be injected directly into the eye or reflected off of the internal wall of the aspiration tube. The handpiece may also contain other tubes, for example, for injecting relatively cool surgical fluid.

Accordingly, one objective of the present invention is to provide a surgical handpiece having at least two tubes.

Another objective of the present invention is to provide a safer tip for a surgical handpiece having a pumping chamber.

Another objective of the present invention is to provide a surgical handpiece having a device for delivering the surgical fluid through the handpiece in pulses that do not directly enter the eye.

Another objective of the present invention is to provide a surgical handpiece having a steerable device for delivering the surgical fluid through the handpiece in pulses.

Another objective of the present invention is to provide a surgical handpiece having a deflector for delivering the surgical fluid through the handpiece in pulses into the eye alternatively directly or indirectly.

These and other advantages and objectives of the present invention will become apparent from the detailed description and claims that follow.

### BRIEF DESCRIPTION OF THE DRAWINGS

FIG. 1 is a front, upper left perspective view of the handpiece of the present invention.

FIG. 2 is a rear, upper right perspective view of the handpiece of the present invention.

FIG. 3 is a cross-sectional view of the handpiece of the present invention taken along a plane passing through the irrigation channel.

FIG. 4 is a cross-sectional view of the handpiece of the present invention taken along a plane passing through the aspiration channel.

FIG. 5 is an enlarged partial cross-sectional view of the handpiece of the present invention taken at circle 5 in FIG. 4.

FIG. 6 is an enlarged partial cross-sectional view of the handpiece of the present invention taken at circle 6 in FIG. 3.

FIG. 7 is an enlarged cross-sectional view of the handpiece of the present invention taken at circle 7 in FIGS. 3 and 4, and showing a resistive boiler pump.

FIG. 8 is a schematic cross-sectional view of a heating element boiler pump that may be used with the present invention.

FIG. 9 is an exploded, partial cross-section view of one embodiment of the handpiece of the present invention.

FIG. 10 is an enlarged cross-sectional view of one alternative tip design for use with the present invention.

FIG. 11 is an enlarged cross-sectional view of a second alternative tip design for use with the present invention showing the flexible tip in a straight configuration.

FIG. 12 is an enlarged cross-sectional view of the second alternative tip design for use with the present invention illustrated in FIG. 11 showing the flexible tip in a curved configuration.

FIG. 13 is an enlarged cross-sectional view of a third alternative tip design for use with the present invention showing the flexible tip in a straight configuration.

FIG. 14 is an enlarged cross-sectional view of the third alternative tip design for use with the present invention illustrated in FIG. 13 showing the flexible tip in a curved configuration.

FIG. 15 is a perspective view of a fourth alternative tip design for use with the present invention showing the slidable nozzle in an extended position.

FIG. 16 is a perspective view of the fourth alternative tip design for use with the present invention illustrated in FIG. 15 showing the nozzle in retracted position.

FIG. 17A is a front plan view of a fifth alternative tip design for use with the present invention showing the rotatable deflector in a straight configuration.

FIG. 17B is a longitudinal cross-sectional view of the fifth alternative tip design for use with the present invention illustrated in FIG. 17.

FIG. 18A is a front plan view of the fifth alternative tip design for use with the present invention showing the rotatable deflector in a rotated configuration.

FIG. 18B is a longitudinal cross-sectional view of the fifth alternative tip design for use with the present invention illustrated in FIG. 18.

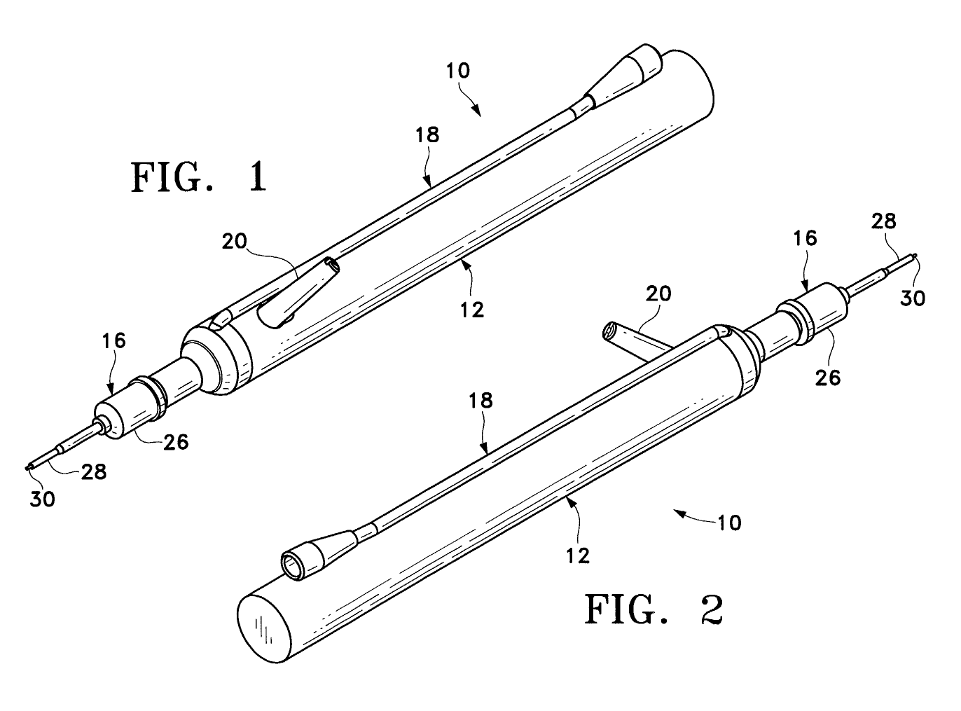

### DETAILED DESCRIPTION OF THE INVENTION

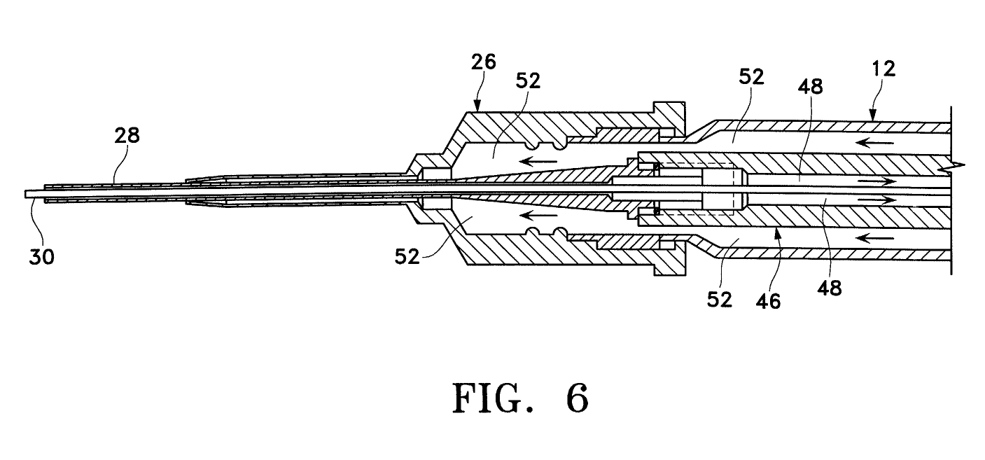

Handpiece 10 of the present invention generally includes handpiece body 12 and operative tip 16. Body 12 generally includes external irrigation tube 18 and aspiration fitting 20. Body 12 is similar in construction to well-known in the art phacoemulsification handpieces and may be made from plastic, titanium or stainless steel. As best seen in FIG. 6, operative tip 16 includes tip/cap sleeve 26, tube 28 and tube 30. Sleeve 26 may be any suitable commercially available phacoemulsification tip/cap sleeve or sleeve 26 may be incorporated into other tubes as a multi-lumen tube. Tube 28 may be any commercially available hollow phacoemulsification cutting tip, such as the TURBOSONICS tip available from Alcon Laboratories, Inc., Fort Worth, Tex. Tube 30 may be any suitably sized tubing to fit within tube 28, for example 29 gauge hypodermic needle tubing. Alternatively, as best seen in FIG. 10, tube 30′ may be external to tube 28′ with a distal tip 27 that terminates within bore 29 of tube 28′ near distal tip 31 of tube 28′. Preferably, tube 30′ is angled at between 25° and 50° and terminates approximately 0.1 mm to 3.0 mm from distal tip 31. Such an arrangement causes fluid exiting tube 28′ to reflect off of internal wall 33 of tube 28′ prior to exiting out of distal tip 31, thereby reducing the intensity of the pressure pulse prior to contact with eye tissue. The intensity of the pressure pulse decays with distance from tip 31; consequently, efficiency is best for tissue that is held at or within tip 31.

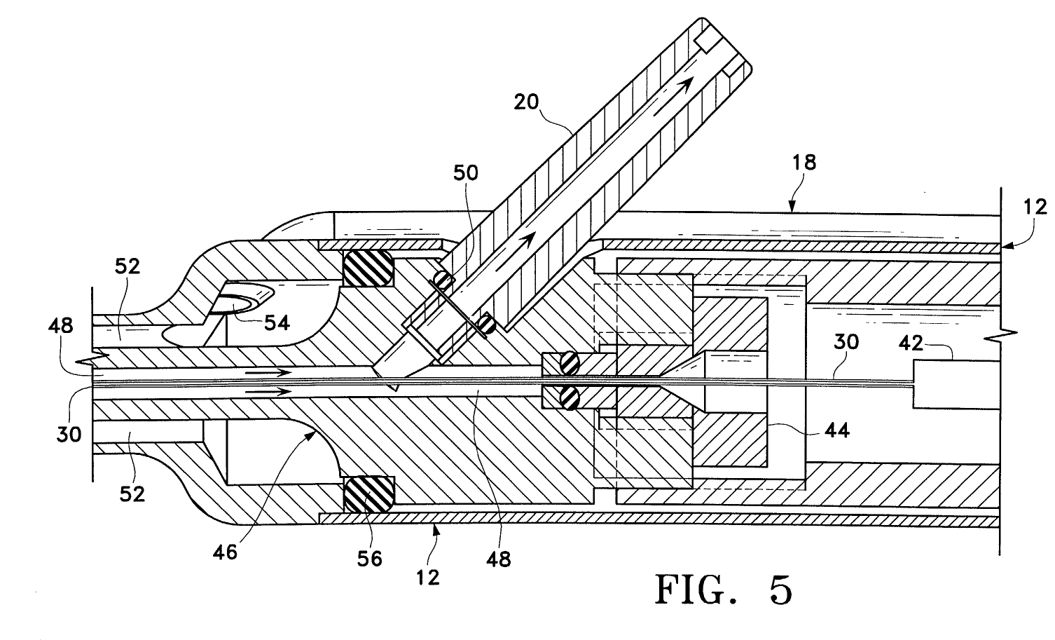

As best seen in FIG. 5, tube 30 is free on the distal end and connected to pumping chamber 42 on the proximal end. Tube 30 and pumping chamber 42 may be sealed fluid tight by any suitable means having a relatively high melting point, such as silver solder. Fitting 44 holds tube 30 within bore 48 of aspiration horn 46. Bore 48 communicates with fitting 20, which is journaled into horn 46 and sealed with O-ring seal 50 to form an aspiration pathway through horn 46 and out fitting 20. Horn 46 is held within body 12 by O-ring seal 56 to form irrigation tube 52 which communicates with irrigation tube 18 at port 54.

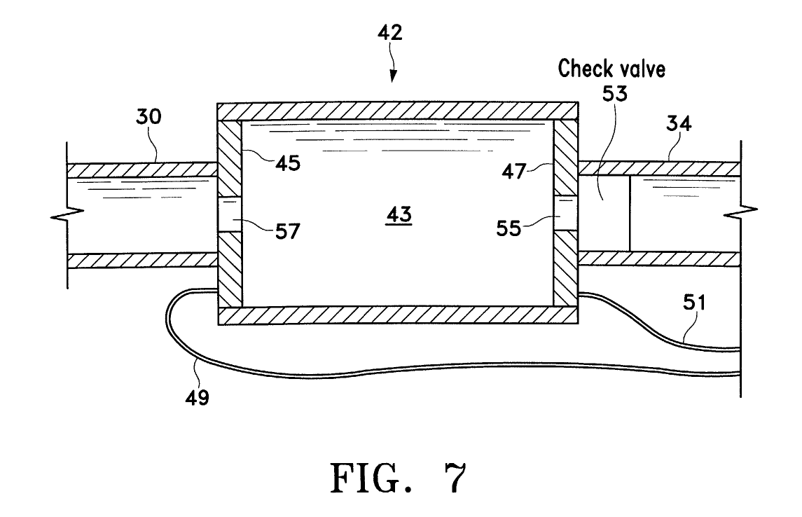

As best seen in FIG. 7, in a first embodiment of the present invention, pumping chamber 42 contains a relatively large pumping reservoir 43 that is sealed on both ends by electrodes 45 and 47. Electrical power is supplied to electrodes 45 and 47 by insulated wires 49 and 51, respectively. In use, surgical fluid (e.g. saline irrigating solution) enters reservoir 43 through port 55, tube 34 and check valve 53, check valves 53 being well-known in the art. Electrical current (preferably Radio Frequency Alternating Current or RFAC) is delivered to and across electrodes 45 and 47 because of the conductive nature of the surgical fluid. As the current flows through the surgical fluid, the surgical fluid boils. As the surgical fluid boils, it expands rapidly out of pumping chamber 42 through port 57 and into tube 30 (check valve 53 prevents the expanding fluid from entering tube 34). The expanding gas bubble pushes the surgical fluid in tube 30 downstream of pumping chamber 42 forward. Subsequent pulses of electrical current form sequential gas bubbles that move surgical fluid down tube 30. The size and pressure of the fluid pulse obtained by pumping chamber 42 can be varied by varying the length, timing and/or power of the electrical pulse sent to electrodes 45 and 47 and by varying the dimensions of reservoir 43. In addition, the surgical fluid may be preheated prior to entering pumping chamber 42. Preheating the surgical fluid will decrease the power required by pumping chamber 42 and/or increase the speed at which pressure pulses can be generated.

While several embodiments of the handpiece of the present invention are disclosed, any handpiece producing adequate pressure pulse force, rise time and frequency may also be used. For example, any suitable handpiece producing a pressure pulse force of between 0.03 grams and 20.0 grams, with a rise time of between 1 gram/sec. and 20,000 grams/sec. and a frequency of between 1 Hz and 200 Hz may be used, with between 20 Hz and 100 Hz being most preferred. The pressure pulse force and frequency will vary with the hardness of the material being removed. For example, the inventors have found that a lower frequency with a higher pulse force is most efficient at debulking and removing the relatively hard nuclear material, with a higher frequency and lower pulse force being useful in removing softer epinuclear and cortical material. Infusion pressure, aspiration flow rate and vacuum limit are similar to current phacoemulsification techniques.

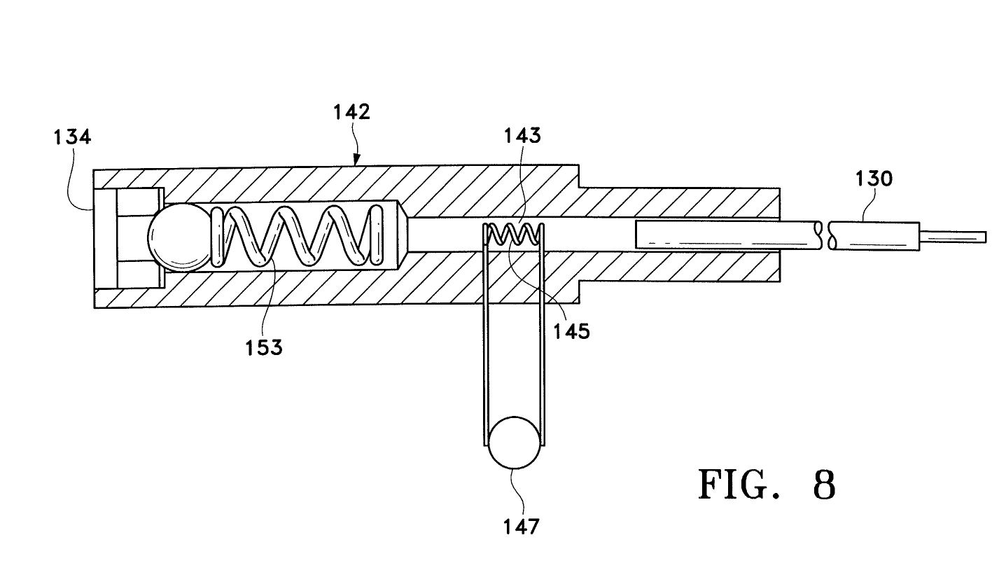

As best seen in FIG. 8, the fluid in reservoir 143 in pumping chamber 142 may also be heated by the use of heating element 145 that is internal to reservoir 143. Heating element 145 may be, for example, a coil of 0.003 inch diameter stainless steel wire which is energized by power source 147. The size and pressure of the fluid pulse obtained by pumping chamber 142 can be varied by varying the length and timing of the electrical pulse sent to element 145 by power source 147 and by varying the dimensions of reservoir 143. The numbers in FIG. 8 are identical to the numbers in FIG. 7 except for the addition of “100” in FIG. 8.

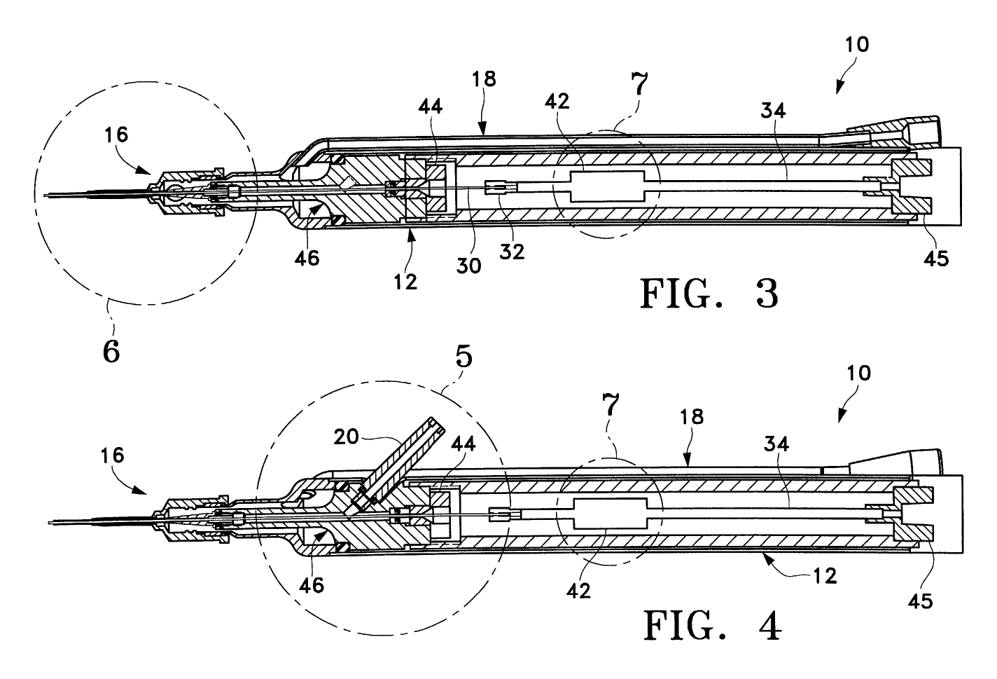

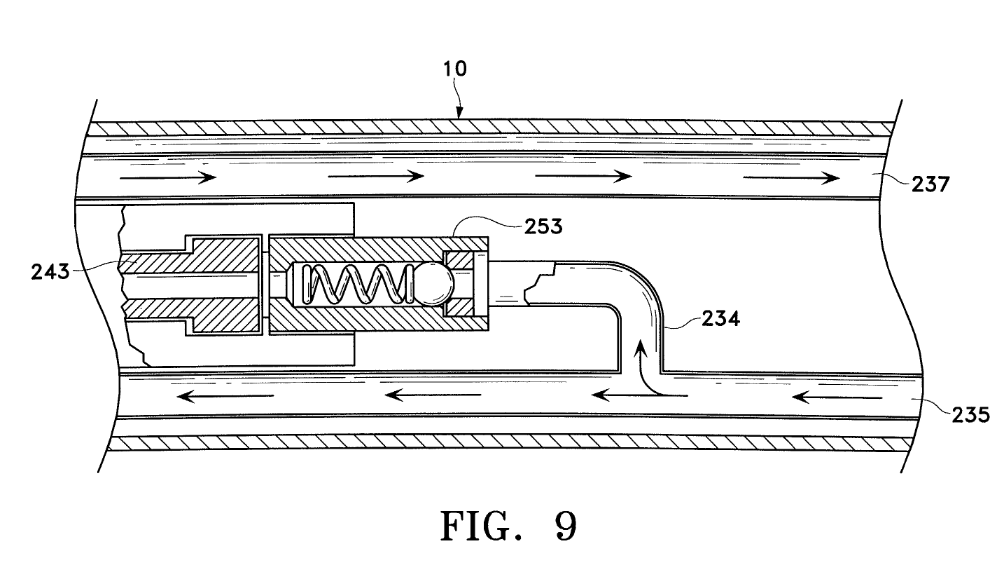

As best seen in FIGS. 3, 4 and 7, surgical fluid may be supplied to pumping chamber 43 through tube 34 or, as seen in FIG. 9, surgical fluid may be supplied to pumping chamber 243 through irrigation fluid tube 234 which branches off main irrigation tube 235 supplying cool surgical fluid to the operative site. As seen in FIG. 9, aspiration tube 237 may be contained internally to handpiece 10. The numbers in FIG. 9 are identical to the numbers in FIG. 7 except for the addition of “200” in FIG. 9.

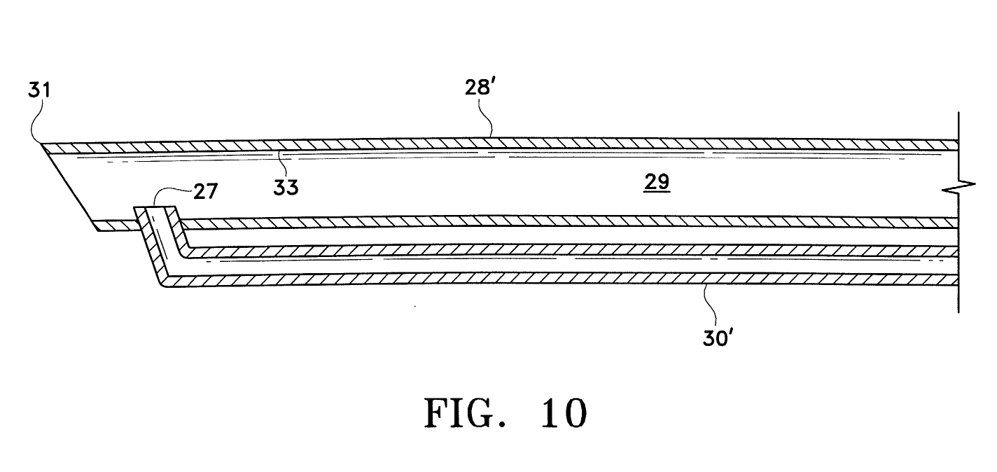

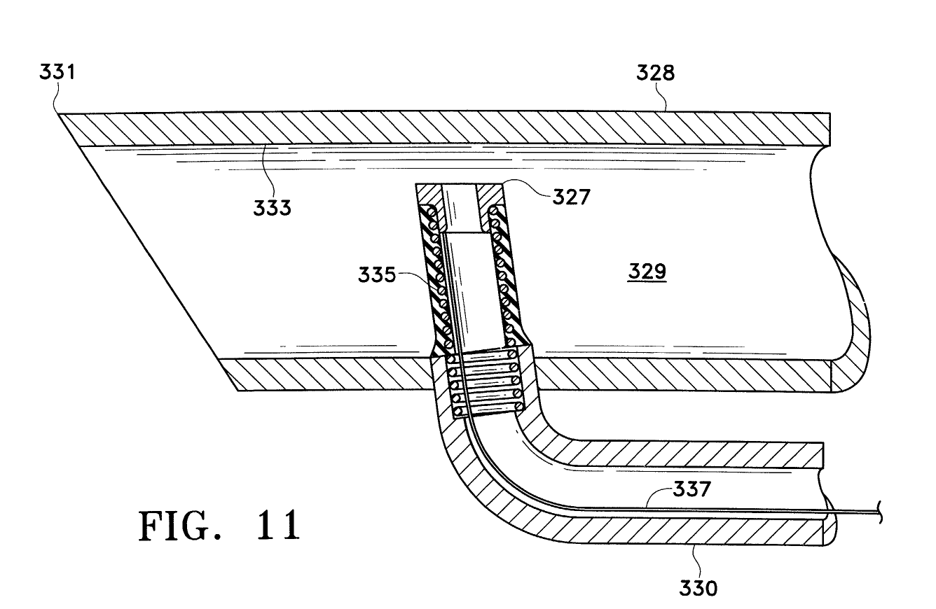

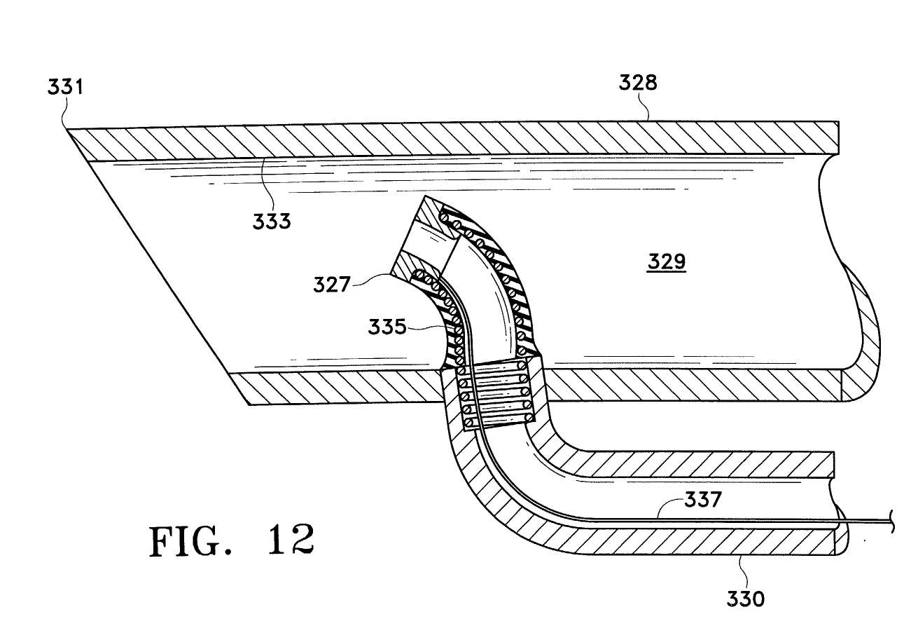

As best seen in FIGS. 11 and 12, in an alternative embodiment of the present invention, distal tip 327 of tube 330 terminates within bore 329 of tube 328 and may be made from an elastomeric covered spring 335 that may be flexed by use of pull wire 337 or other suitable method well-known in the art. For example, U.S. Pat. Nos. 4,921,482, 4,998,916, 5,037,391, 5,108,368, 5,203,772, 5,308,324, 5,372,587, 5,378,234 (all to Hammerslag, et al.) and 5,217,465 (Steppe), the entire contents of which being incorporated herein by reference, all disclose method of steering a flexible tube of similar construction as the present invention. As a result, the pressure pulse exiting tip 327 may be directed against internal wall 333 of tube 328 (as shown in FIG. 11) or directed out tip 331 (as shown in FIG. 12).

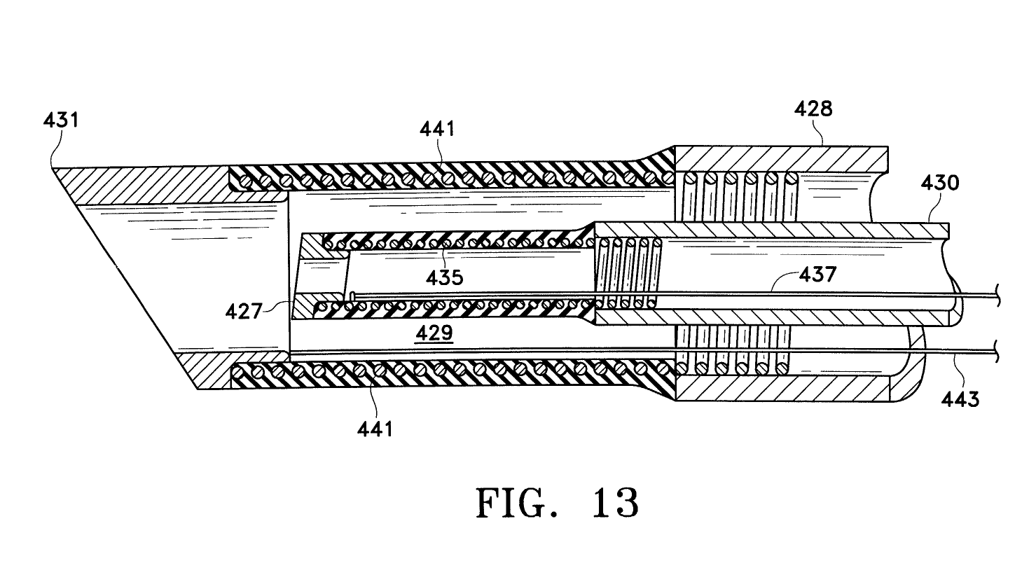

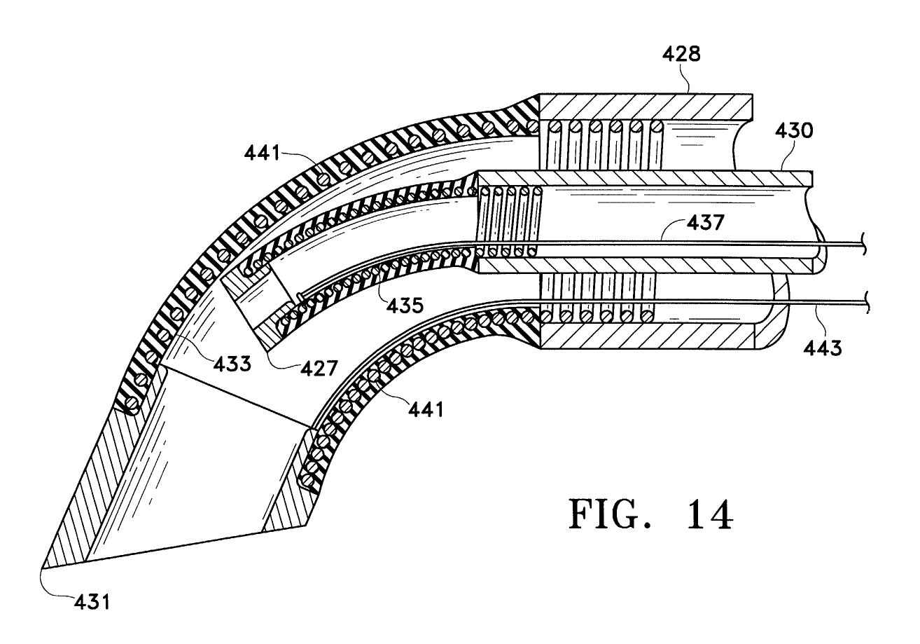

As best seen in FIGS. 13 and 14, in yet another alternative embodiment of the present invention similar to the embodiment illustrated in FIGS. 11 and 12, tube 430 may be coaxial with tube 428 and contained within bore 429 of tube 428. Distal tip 427 of tube 430 and distal tip 439 of tube 428 may be made from an elastomeric covered springs 435 and 441, respectively that may be flexed by use of pull wires 437 and 443, respectively. As a result, the pressure pulse exiting tip 427 may be directed against internal wall 433 of tube 428 (as shown in FIG. 14) or directed out tip 431 (as shown in FIG. 13) and tip 431 may be steered within the eye as required, as shown in FIG. 14. Alternativley, tube 430 may be attached to tube 428 so that tube 430 flexes with tube 428, thereby eliminating the need for pull wire 437. Tube 430 may have any fixed orientation desired with respect to tube 428.

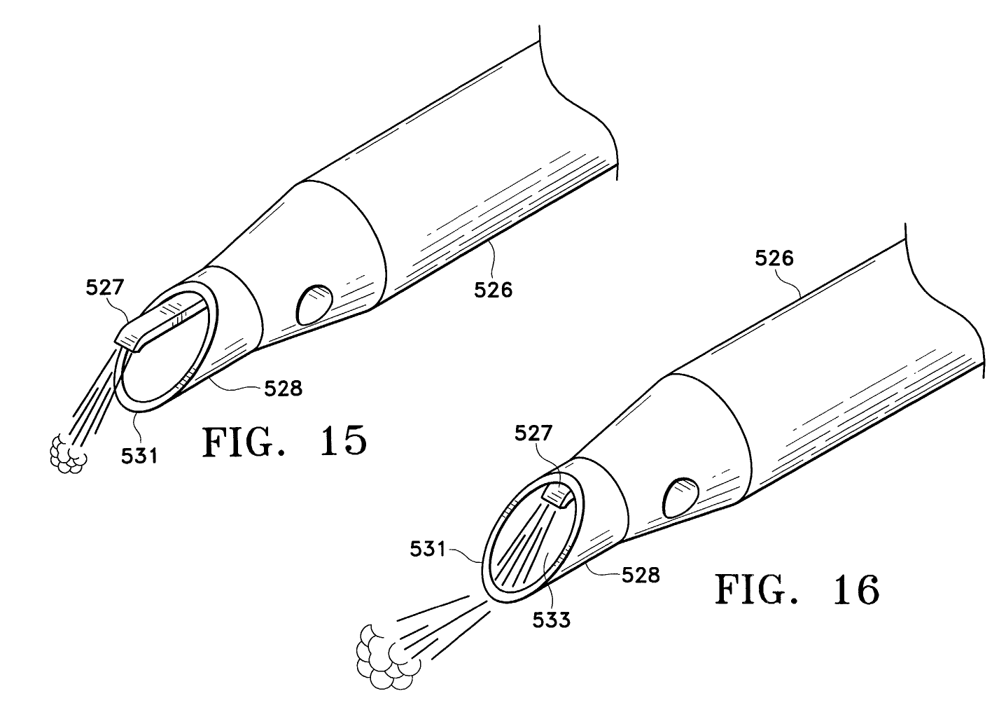

In still another embodiment of the present invention, illustrated in FIGS. 15 and 16, distal tip 527 is slidably received on the end of the heated fluid injection tube (not shown), and may be extended out of tip 531, as shown in FIG. 15, or retracted into tip 531, as shown in FIG. 16. In the extended position, tip 527 directs the heated pressure pulses directly at the targeted tissue. In the retracted position, tip 527 directs the heated pressure pulses against internal wall 533 of tube 528. Suitable methods for extending and retracting tip 527 include the pull wires illustrated in FIGS. 11-14 and 17A-18B (not shown).

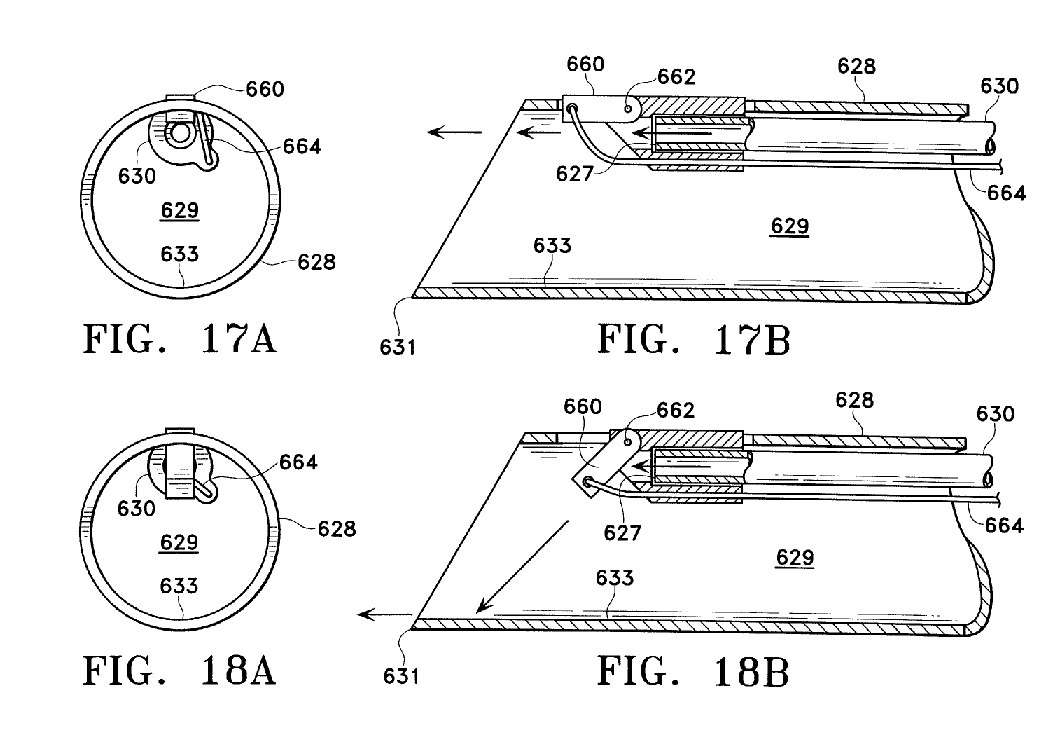

As best seen in FIGS. 17A-18B, in yet another embodiment of the present invention, tube 630 is contained within bore 629 of tube 628 so that tip 627 terminates internal to bore 629. Deflector 660 attaches to tube 628 near tip 631 just outside tip 627 so that deflector 660 rotates about pivot pin 662 by the urging of control wire 664. As seen in FIGS. 17A and 17B, pushing forward (toward tip 631) on wire 664 rotates deflector 660 clockwise about pin 662, thereby rotating deflector 660 relatively clear of fluid exiting tip 627 of tube 630 and allowing the fluid to flow out of tip 627 relatively freely and contact directly the targeted tissue. Alternatively, the force of the pressure pulse may be used to rotate deflector 660 about pin 662. As seen in FIGS. 18A-18B, pulling on wire 664 (away from tip 631) causes deflector 660 to rotate counterclockwise about pin 662, thereby partially deflecting fluid exiting tip 627 of tube 630 and directing the fluid against internal wall 633 of tube 628.

Any of a number of methods can be employed to limit the amount of heat introduced into the eye. For example, the pulse train duty cycle of the heated solution can be varied so that the total amount of heated solution introduced into the eye does not vary with the pulse frequency. Alternatively, the aspiration flow rate can be varied as a function of pulse frequency so that as pulse frequency increases aspiration flow rate increases proportionally.

This description is given for purposes of illustration and explanation. It will be apparent to those skilled in the relevant art that changes and modifications may be made to the invention described above without departing from its scope or spirit. For example, it will be recognized by those skilled in the art that the present invention may be combined with ultrasonic and/or rotating cutting tips to enhance performance.

We claim:

1. A liquefracture handpiece, comprising:
a) a body having an aspiration tube; and
b) an irrigation tube having a flexible distal tip, the irrigation tube being external to the aspiration tube and the distal tip terminating internal to the aspiration tube.

2. The handpiece of claim 1 further comprising a pumping chamber contained within the body and fluidly connected to the irrigation tube.

3. The handpiece of claim 1 wherein a distal portion of the aspiration tube is flexible.

4. The handpiece of claim 2 wherein the pumping chamber produces pressure pulses with a force of between 0.03 grams and 20.0 grams.

5. The handpiece of claim 2 wherein the pumping chamber produces pressure pulses with a rise time of between 1 gram/second and 20,000 grams/second.

6. A liquefracture handpiece, comprising:
a) a body having an aspiration tube; and
b) an irrigation tube having a flexible distal tip, the irrigation tube being internal to the aspiration tube.

7. The handpiece of claim 6 further comprising a pumping chamber contained within the body and fluidly connected to the irrigation tube.

8. The handpiece of claim 6 wherein a distal portion of the aspiration tube is flexible.

9. The handpiece of claim 7 wherein the pumping chamber produces pressure pulses with a force of between 0.03 grams and 20.0 grams.

10. The handpiece of claim 7 wherein the pumping chamber produces pressure pulses with a rise time of between 1 gram/second and 20,000 grams/second.

11. A liquefracture handpiece, comprising:
a) a body having an aspiration tube; and
b) an irrigation tube having an extendable distal tip, the irrigation tube being internal to the aspiration tube.

12. The handpiece of claim 11 further comprising a pumping chamber contained within the body and fluidly connected to the irrigation tube.

13. The handpiece of claim 12 wherein the pumping chamber produces pressure pulses with a force of between 0.03 grams and 20.0 grams.

14. The handpiece of claim 12 wherein the pumping chamber produces pressure pulses with a rise time of between 1 gram/second and 20,000 grams/second.

15. A liquefracture handpiece, comprising:
a) a body having an aspiration tube;
b) an irrigation tube having a distal tip, the irrigation tube being internal to the aspiration tube; and
c) a deflector located at the distal tip and movable so as to alternatively deflect fluid existing the distal tip or allow fluid exiting the distal tip to flow relatively freely.

16. The handpiece of claim 15 further comprising a pumping chamber contained within the body and fluidly connected to the irrigation tube.

17. The handpiece of claim 16 wherein the pumping chamber produces pressure pulses with a force of between 0.03 grams and 20.0 grams.

18. The handpiece of claim 16 wherein the pumping chamber produces pressure pulses with a rise time of between 1 gram/second and 20,000 grams/second.
